(19) United States
(12) Patent Application Publication
(10) Pub. No.: US 2012/0121547 A1
(43) Pub. Date: May 17, 2012

(54) Methods and compositions for treating chronic wounds

(76) Inventors: Donald J. Wagner, Venetia, PA (US); Linda O. Palladino, Stormville, NY (US)

(21) Appl. No.: 13/199,915

(22) Filed: Sep. 13, 2011

Related U.S. Application Data
(60) Provisional application No. 61/403,452, filed on Sep. 16, 2010.

(51) Int. Cl. A61K 35/48; A61P 17/02

(52) U.S. Cl. 424/93.7

(57) ABSTRACT

The invention is directed to novel methods for treating wounds, in particular, chronic wounds such as diabetic ulcers. Such methods utilize novel cell and cell-product compositions in combination with insulin.

### CROSS-REFERENCE TO RELATED APPLICATIONS

This application claims priority under 35 USC §119(e) to U.S. Provisional Application No. 61/403,452, filed Sep. 16, 2010, the contents of which is incorporated herein by reference in its entirety.

### FIELD OF THE INVENTION

The field of the invention is directed to novel methods for treating wounds, in particular, chronic wounds such as diabetic ulcers. Such methods utilize novel cell and cell-product compositions in combination with insulin. Such novel cell and cell-product compositions include, but are not limited to, Extraembryonic Cytokine Secreting cells (herein referred to as ECS cells), conditioned media derived therefrom (herein referred to as ECS cell-conditioned medium or ECS cell-CM), Amnion-derived Multipotent Progenitor cells (herein referred to as AMP cells), and conditioned media derived therefrom (herein referred to as Amnion-derived Cellular Cytokine Solution or ACCS), as well as cell lysates and cell products derived from ECS cells and AMP cells.

### DESCRIPTION OF RELATED ART

Greenway, S. E., et al, (J Wound Care, 1999 Nov; 8(10):526-8) report the results obtained in a randomized, double-blind, placebo-controlled clinical trial evaluating topical insulin in wound healing. The authors conclude that topical insulin accelerates wound healing in humans.

Zhang, X. J., et al, (J Surg Res, 2007 Sep; 42(1):90-6) report that local insulin-zinc injection accelerates skin donor site wound healing.

Zhang, X. J., et al, (Wound Repair Regen, 2007 Mar-Apr; 15(2):258-65) report that local insulin-zinc injection stimulates wound DNA synthesis, which would be expected to accelerate wound reepithelialization.

Duckworth, W. C., et al, J Clin Endo & Met 2004, 89(2):847-851) describe the insulin-degrading activity in wound fluid in both non-diabetic and diabetic subjects. The authors conclude that insulin-degrading activity is greater in the wound fluid of diabetics than non-diabetics and that this negatively impacts the healing ability of diabetics.

U.S. Publication Nos. 20060222634 and 20070231297 describe inter alia a novel population of cells purified from the amnion (referred to herein as AMP cells), novel compositions derived from such cells (described herein as ACCS), as well as uses of the cells and cell-derived compositions to accelerate healing of both acute and chronic wounds, including chronic infected wounds.

### BACKGROUND OF THE INVENTION

A chronic wound is a wound that does not heal in the orderly phases of wound healing or in a timely fashion the way most other wounds do. Chronic wounds appear to be stalled or arrested in one or more of the phases of wound healing. For example, chronic wounds often remain in the inflammatory phase for too long. In acute wounds, a precise balance exists between the production and degradation of important molecules such as collagen. However, in chronic wounds this balance is disrupted and degradation plays too large a role. Chronic wounds may take months or years to heal or may never heal at all. These wounds cause patients severe emotional and physical stress as well as creating a significant financial burden on patients and the entire healthcare system.

Acute and chronic wounds are at opposite ends of a spectrum of wound healing types that progress toward being healed at significantly different rates. The vast majority of chronic wounds can be classified into four categories: venous ulcers, diabetic ulcers, pressure ulcers and sickle cell ulcers. A small number of chronic wounds that do not fall into one of these four categories may be due to causes such as radiation poisoning or ischemia.

While treatment of the different chronic wound types varies somewhat, appropriate treatment seeks to address the problems at the root of chronic wounds, including inflammation, ischemia, bacterial load, and imbalance of proteases. Various methods exist to ameliorate these problems, including antibiotic and antibacterial use, debridement, irrigation, vacuum-assisted closure, warming, oxygenation, moist wound healing, removal of mechanical stress, and adding cells or other materials to secrete or enhance levels of healing factors.

Clearly, the lack of methods and compositions to satisfactorily promote suitable healing of chronic wounds represents a significant unmet medical need which the present invention seeks to fulfill.

### BRIEF SUMMARY OF THE INVENTION

It is an object of the instant invention to provide novel methods and compositions for treating chronic wounds such as venous ulcers, diabetic ulcers, pressure ulcers and sickle cell ulcers. Such novel methods and compositions provide a means for accelerating the healing of chronic wounds, in particular diabetic ulcers, whose healing is typically impeded by inflammation and infection, and often other factors as well. Specifically, diabetic ulcers exhibit high glucose levels in the wound fluid and this has been shown to exacerbate inflammation of the wound, thus interfering with proper healing. The novel compositions described herein work synergistically with insulin to 1) lower glucose concentration in the wound fluid, thus reducing inflammation and 2) allowing the physiologically relevant wound healing factors secreted at physiologic levels by the novel cells described herein to accelerate chronic wound healing, even infected wounds. This unique “combination composition” represents a new approach in the treatment of chronic wounds.

The novel combination compositions used in the methods of the invention include, but are not limited to, extraembryonic cytokine secreting cells (herein referred to as ECS cells), conditioned media derived therefrom (herein referred to as ECS cell conditioned medium or ECS cell-CM), Amnion-derived Multipotent Progenitor cells (herein referred to as AMP cells), and conditioned media derived therefrom (herein referred to as Amnion-derived Cellular Cytokine Solution or ACCS), as well as cell lysates and cell products derived from the cells, each in combination with insulin (i.e. ECS cells/insulin; ECS cell-CM /insulin; ECS cell lysates/insulin; ECS cell products/insulin; AMP cells/insulin; ACCS/insulin; AMP cell lysates/insulin; AMP cell products/insulin, etc.), or in combination with each other as well as with insulin (i.e. ECS cells/ECS cell-CM/ insulin, etc.).

Accordingly, a first aspect of the invention is a method of accelerating the rate of healing of a chronic wound in a subject in need thereof comprising applying to the chronic wound a combination composition selected from the group consisting of a combination composition comprising Extraembryonic Cytokine-Secreting (ECS) cells, insulin, and a carrier; a combination composition comprising conditioned media derived from ECS, insulin, and a carrier; a combination composition comprising Amnion-derived Multipotent Progenitor (AMP) cells, insulin, and a carrier; a combination composition, and a combination comprising Amnion-derived Cellular Cytokine Solution (ACCS), insulin, and a carrier, wherein the chronic wound is selected from the group consisting of a diabetic ulcer, a venous ulcer, a pressure ulcer, and a sickle cell ulcer.

A second aspect of the invention is a method of lowering glucose concentrations in chronic wound fluid in a subject in need thereof comprising applying to the chronic wound a combination composition selected from the group consisting of a combination composition comprising Extraembryonic Cytokine-Secreting (ECS) cells, insulin, and a carrier; a combination composition comprising conditioned media derived from ECS, insulin, and a carrier; a combination composition comprising Amnion-derived Multipotent Progenitor (AMP) cells, insulin, and a carrier; a combination composition, and a combination comprising Amnion-derived Cellular Cytokine Solution (ACCS), insulin, and a carrier, wherein the chronic wound is selected from the group consisting of a diabetic ulcer, a venous ulcer, a pressure ulcer, and a sickle cell ulcer.

Specific embodiments of aspects one and two are those wherein the insulin in the composition is added to the composition; wherein the insulin in the composition is added to the composition by ECS cells or AMP cells which have been genetically modified to secrete insulin; wherein the insulin in the composition is added to the composition by combining ECS cells or AMP cells with insulin-secreting islet cells or insulin-secreting islet-like cells; or wherein the combination composition is selected from the group consisting of: Composition A: ECS cells+GP-AMP cells; Composition B: ECS cells+GP-AE cells; Composition C: AMP cells+GP-AMP cells; Composition D: AMP cells+GP-AE cells; Composition E: ECS conditioned media+GP-AMP cells; Composition F: ECS conditioned media+GP-AE cells; Composition G: ACCS+GP-AMP cells; and Composition H: ACCS+GP-AE cells.

Other features and advantages of the invention will be apparent from the accompanying description, examples and the claims. The contents of all references, pending patent applications and published patents, cited throughout this application are hereby expressly incorporated by reference. In case of conflict, the present specification, including definitions, will control.

### Definitions

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cells” or “EE cells” means a population of cells derived from the extraembryonic tissue. EE cells may be selected from populations of cells and compositions described in this application and in U.S.2003/0235563, U.S.2004/0161419, U.S.2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, U.S.2006/0078993, PCT/US00/40052, U.S. Pat. No. 7,045,148, U.S.2004/0048372, and U.S.2003/0032179, the contents of which are incorporated herein by reference in their entirety.

As used herein, the term “Amnion-derived Multipotent Progenitor cell” or “AMP cell” means a novel population of cells that are selected from the epithelial cells derived from the amnion and cultured under very specific conditions. AMP cells have the following characteristics. They have not been cultured in the presence of any non-human animal materials, making them and cell products derived from them suitable for human clinical use as they are not xeno-contaminated. AMP cells are cultured in basal medium supplemented with human serum albumin. In a preferred embodiment, the AMP cells secrete the cytokines VEGF, Angiogenin, PDGF and TGFβ2 and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2. The AMP cells may optionally express Thymosin β4. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are isolated and cultured, will not react with antibodies to the stem/progenitor cell markers c-kit (CD117) and Thy-1 (CD90). Several procedures used to obtain cells from full term or pre-term placenta are known in the art (see, for example, U.S. 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

As used herein, the term “primed” refers to cells which are exposed to conditions that bias them towards a desired phenotype (i.e. high glucose exposure in utero biases amnion epithelial cells towards a pancreatic cell phenotype).”

As used herein, the term “pregnant diabetic woman” means a pregnant woman who has a condition which causes her blood sugar to be above normal homeostatic ranges. Examples of such conditions include but are not limited to hyperglycemia, Type I diabetes, Type II diabetes, gestational diabetes, metabolic syndrome, and the like.

As used herein, the term “glucose-primed extraembryonic cytokine secreting cells” or “GP-ECS cells” means a substantially purified population of cells derived from the extraembryonic tissues delivered by a pregnant diabetic woman (either vaginally or via cesarean section) which, in addition to having the characteristics of ECS cells described above, possess additional characteristics. For example, such cells are exposed to high glucose levels in utero and thus have the further characteristic of being primed for differentiation into endoderm, in particular pancreatic endoderm, to function as islet-like cells (i.e. exhibit incremental glucose-dependent insulin secretion), and express markers associated with islet-like cells and/or pancreatic endoderm.

As used herein, the term “glucose-primed Amnion-derived Multipotent Progenitor cells” or “GP-AMP cells” means a substantially purified population of cells that are selected from the amnion. In addition to having the characteristics of AMP cells described above, GP-AMP cells possess additional characteristics. For example, such cells are exposed to high glucose levels in utero and thus have the further characteristic of being primed for differentiation into endoderm, in particular pancreatic endoderm, to function as islet-like cells (i.e. exhibit incremental glucose-dependent insulin secretion), and express markers associated with islet-like cells and/or pancreatic endoderm.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “expanded”, in reference to cell compositions, means that the cell population constitutes a significantly higher concentration of cells than is obtained using previous methods.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, the term “differentiation” means the process by which cells become progressively more specialized. As used herein, the term “differentiation efficiency” means the percentage of cells in a population that are differentiating or are able to differentiate.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. 6,372,494 which is incorporated by reference in its entirety herein.

As used herein, the term “Amnion-derived Cellular Cytokine Solution” or “ACCS” means conditioned medium that has been derived from AMP cells or expanded AMP cells that have been cultured in basal media supplemented with human serum albumin. Amnion-derived cellular cytokine solution or ACCS has previously been referred to as “amnion-derived cytokine suspension”.

As used herein, the term “glucose-primed ECS cell conditioned media” or “GP-ECS-CM” means conditioned medium that has been derived from GP-ECS cells or expanded GP-ECS cells. A specific embodiment is “glucose-primed Amnion-derived Cellular Cytokine Solution” or “GP-ACCS” which is derived from GP-AMP cells.

The term “physiological level” as used herein means the level that a substance in a living system is found and that is relevant to the proper functioning of a biochemical and/or biological process.

The term “therapeutically effective amount” means that amount of a therapeutic agent necessary to achieve a desired physiological effect (i.e. control blood glucose levels).

As used herein, the term “temporal expression” means expression of a gene or protein which is limited in time, temporary, or transient.

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells that are either in an undifferentiated, partially differentiated, or fully differentiated form, or a combination thereof, into a human or other animal.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “topical administration” means the delivery of a medication by application to the skin or mucous membrane.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e. contusion, penetrating), thermal, chemical, electrical, concussive and incisional injuries; elective injuries such as operative surgery and resultant incisional hernias, fistulae, etc.; acute wounds, chronic wounds, infected wounds, and sterile wounds, as well as wounds associated with disease states (i.e. ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scarring and/or the wounded area possesses tissue strength that is closer to that of uninjured tissue and/or the wounded tissue attains some degree of functional recovery.

As used herein, the term “clustered glucose-primed ECS cell compositions” or “clustered glucose-primed AMP cell compositions” refers to cell compositions wherein at least 50% and up to about 95% of the cells form clusters.

“Pancreatic progenitor cell” as defined herein is a cell which can differentiate into a cell of pancreatic lineage, e.g., a cell which can produce a hormone or enzyme normally produced by a pancreatic cell. For instance, a pancreatic progenitor cell may be caused to differentiate, at least partially, into alpha, beta, delta, or phi islet cells, or a cell of exocrine fate. In accordance with the method of the invention, the pancreatic progenitor cells of the invention can be cultured prior to administration to a subject under conditions which promote cell proliferation and/or differentiation. These conditions include but are not limited to culturing the cells to allow proliferation in vitro at which time the cells may form pseudo islet-like spheroids and secrete insulin, glucagon, and somatostatin.

The term “islet-like cell” as used herein means having some but not necessarily all of the characteristics of one of the cell types (α, β, γ or δ) present in a mature pancreatic islet. The islet-like cell will express only one of the following pancreatic endocrine cell hormones: Insulin, glucagon, Somatostatin, Pancreatic Polypeptide.

The term “islet-like structures” as defined herein are structures containing islet-like cells. Islet-like structures refers to the spheroids of cells derived from the methods of the invention which take on both the appearance of pancreatic α, β, γ or δ cells, as well as their function. Their coordinated function includes the ability to respond to glucose.

As used herein, the term “spheroid” or “spheroids” means multicellular clusters in suspension cultures.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, the preferred methods and materials are now described.

It must be noted that as used herein and in the appended claims, the singular forms “a,” “and” and “the” include plural references unless the context clearly dictates otherwise.

### Therapeutic Uses

It has been discovered that the AMP cells and ACCS described herein are able to accelerate the rate of wound healing (including increased wound strength) in both non-contaminated and contaminated (infected) wounds (see U.S. Publication No. 20060222634 and 20070231297, incorporated herein in their entirety) as compared to non-treated wounds. It is long known in the art that infected wounds either do not heal or the rate of healing is very slow. However, using the novel compositions, the rate of wound healing is accelerated compared to untreated even when the wound is infected. This unique ability to heal the wounds in the face of infection is not based on any antibacterial effect of the compositions, but rather is due to the unique combination of physiologically relevant cytokines secreted by the cells at physiological levels and the unique conditions in which the cells are cultured.

The secretion of these physiologically relevant cytokines may be into the extracellular space in which they are placed or into culture media to form ACCS. Such physiologically relevant cytokines include VEGF, PDGF, Angiogenin, TGFβ2, TIMP-1 and TIMP-2. Because the effectiveness of the AMP cells and ACCS compositions are due to this unique cytokine profile, it is believed that any ECS cell or cell-derived composition that produces a comparable cytokine profile will be equally effective, provided such cells are cultured in accordance with the unique conditions described herein.

The cytokines described above are known to be involved in many physiological processes including wound healing. VEGF and Angiogenin are both involved in regulating angiogenesis and vascularization. PDGF is involved in regulating cell growth and division and, like VEGF and Angiogenin, plays a significant role in angiogenesis. TGFβ2 is a member of the TGF superfamily, a group of cytokines that play a number of different roles in many cellular functions. TIMP-1 and TIMP-2 are tissue inhibitors of metalloproteinases (MMPs). MMPs are a family of inflammatory cytokines that are present in high levels in non-healing wounds (such as chronic wounds) and are thought to interfere with wound healing by destroying cytokines and other proteins essential to the wound healing process. Previous studies have demonstrated that the ratio of MMP-9 to TIMP-1 in wound fluids is inversely correlated with the healing of pressure wounds (Ladwig, G P, et al. Wound Rep Reg 2002, 10:26-37). As described in U.S. Publication No. 20070231297 the physiologically relevant levels of TIMP-1 and TIMP-2 secreted by AMP cells and found in ACCS block MMP activity and thus promote accelerated wound healing as compared to untreated wounds.

It has been reported in the literature that insulin added to a wound is able to promote wound healing (see for example Greenway, S. E., et al, J Wound Care, 1999 Nov; 8(10):526-8, who conclude that topical insulin accelerates wound healing in humans; Zhang, X. J., et al, J Surg Res, 2007 Sep; 42(1):90-6 who report that local insulin-zinc injection accelerates skin donor site wound healing; and Zhang, X. J., et al, Wound Repair Regen, 2007 Mar-Apr; 15(2):258-65) who report that local insulin-zinc injection stimulates wound DNA synthesis, which would be expected to accelerate wound reepithelialization). Thus, a treatment combining the wound healing capabilities of ECS cells, ECS cell-CM, AMP cells, or ACCS in combination with insulin is expected to work synergistically to promote accelerated healing as compared to non-treated, especially in chronic wounds.

### Types of Chronic Wounds

Diabetic Ulcers: Diabetic ulcers are increasing in prevalence. Diabetics have a 15% higher risk for amputation than the general population due to chronic ulcers. Diabetes causes neuropathy, which inhibits nociception and the perception of pain. Thus patients may not initially notice small wounds to legs and feet, and may therefore fail to prevent infection or repeated injury. Further, diabetes causes immune compromise and damage to small blood vessels, preventing adequate oxygenation of tissue, which can cause chronic wounds. Pressure also plays a role in the formation of diabetic ulcers. The combination compositions and methods of the invention are suitable for treating diabetic ulcers.

Venous Ulcers: Venous ulcers, which usually occur in the legs, account for about 70% to 90% of chronic wounds and mostly affect the elderly. They are thought to be due to venous hypertension caused by improper function of valves that exist in the veins to prevent blood from flowing backward. Ischemia results from the dysfunction and, combined with reperfusion injury, causes the tissue damage that leads to the wounds. The combination compositions and methods of the invention are suitable for treating venous ulcers.

Pressure Ulcers: Pressure ulcers are another leading type of chronic wounds. Pressure ulcers usually occur in people with paralysis which inhibits movement of body parts that are commonly subjected to pressure such as the heels, shoulder blades, and the sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue. The combination compositions and methods of the invention are suitable for treating pressure ulcers.

Sickle Cell Ulcers: Sickle cell ulcers, usually occurring on the leg, are a common complication associated with sickle cell disease. Sickle cell ulcers are more common in men and in patients over 20. It has been reported that 8% to 10% of sickle cell patients develop leg ulcers between the ages of 10 and 50 and as many as 75% of adults with sickle cell anemia who live in tropical areas are affected. The ulcers commonly occur over the lateral or medial aspect of the ankle, and occasionally higher on the shin. Specific characteristics accompany sickle cell ulcers. Pathophysiologic changes which accompany sickle cell ulcers include vessel obstruction by sickled red blood cells that lead to increased venous and capillary pressure, decreased oxygen-carrying capacity of the blood, and interference with proper nutrition and metabolism of cells. The combination compositions and methods of the invention are suitable for treating sickle cell ulcers.

In accordance with the subject invention, a combination composition treatment comprising novel cells (i.e. ECS cells, AMP cells, GP-ECS cells, GP-AMP cells), conditioned medium derived from the cells (i.e. ACCS or GP-ACCS), or cell lysates or cell products derived from the cells, used in combination with insulin, may work synergistically to promote accelerated wound healing, and in particular, the accelerated healing of chronic wounds. This may be especially true in diabetic ulcers, in which glucose levels in wound fluid are high, thus promoting inflammation and inhibiting healing. Insulin has also been shown to drive VEGF release by keratinocytes (Goren, I., et al., J Invest Dermatol, 2008 Jul 31, epub ahead of print), which may lead to increased vascularization and accelerated healing of chronic wounds such as diabetic, pressure, venous and sickle cell ulcers.

Obtaining and Culturing Cells

Various methods for isolating cells from the extraembryonic tissue, which may then be used to produce the ECS cells of the instant invention are described in the art (see, for example, U.S.2003/0235563, U.S.2004/0161419, U.S.2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, U.S.2006/0078993, PCT/US00/40052, U.S. Pat. No. 7,045,148, U.S.2004/0048372, and U.S.2003/0032179).

Identifying ECS cells—Once extraembryonic tissue is isolated, it is necessary to identify which cells in the tissue have the characteristics associated with ECS cells (see definition above). For example, cells are assayed for their ability to secrete a unique combination of cytokines into the extracellular space or into surrounding culture media. Suitable cells are those in which the cytokine or cytokines occurs in the physiological range of ˜5.0-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2. Suitable cells may optionally secrete Thymosin β4.

AMP cells—AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human serum albumin (and no non-human animal protein); d) selecting AMP cells from the epithelial cell culture, and optionally e) further proliferation of the cells, optionally using additional additives and/or growth factors (i.e. recombinant human EGF). Details are contained in U.S. Publication No. 2006-0222634-A1, which is incorporated herein by reference.

Culturing of the cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Preferably, the medium is IMDM highly enriched basal medium. Such media should either contain human protein or be supplemented with human protein. As used herein a “human protein” is one that is produced naturally or one that is produced using recombinant technology. In specific embodiments, the basal media is IMDM highly enriched basal medium and the human protein is human serum albumin at a concentration of at least 0.5% and up to 10%. In particular embodiments, the human serum albumin concentration is from about 0.5 to about 2%. The human serum albumin may come from a liquid or a dried (powder) form and includes, but is not limited to, recombinant human serum albumin, PLASBUMIN® normal human serum albumin and PLASMANATE® human blood fraction (both made by Talecris Biotherapeutics). In a most preferred embodiment, the cells are cultured using a system that is free of non-human animal products or exposure to eliminate xeno-contamination.

Optionally, other factors are used. In one embodiment, human recombinant epidermal growth factor (hrEGF) at a concentration of between 0-1 μg/mL is used. In a preferred embodiment, the hrEGF concentration is around 10-20 ng/mL. All supplements are clinical grade.

In a specific embodiment, the following method is used to obtain selected AMP cells. The cells are plated into plastic tissue culture vessels (i.e. T75 flasks) immediately upon isolation from the amnion. After ˜1-5 days, preferably ˜1-3 days, and most preferably ˜2 days in culture, non-adherent cells are removed from the plastic tissue culture vessel and discarded and the adherent cells are kept. This attachment of cells to a plastic tissue culture vessel is the selection method used to obtain the desired population of AMP cells. Adherent and non-adherent AMP cells appear to have similar cell surface marker expression profiles but the adherent cells have the advantage of possessing greater viability than the non-adherent population of cells and are thus the desired population of AMP cells. Adherent AMP cells are cultured until they reach ˜13,000-700,000 cells/cm², preferably ˜53,000-500,000 cells/cm² and most preferably ˜120,000-300,000 cells/cm². At this point, the cultures are confluent or close to confluent. Suitable cells cultures will reach this number of cells between ˜5-14 days, preferably between 5-9 days. Attaining this criterion is an indicator of the proliferative potential of the AMP cells and cells that do not achieve this criterion are not selected for further analysis and use. Once the AMP cells reach ˜13,000-700,000 cells/cm², preferably ˜53,000-500,000 cells/cm² and most preferably ˜120,000-300,000 cells/cm², they are removed from the plastic tissue culture vessel and cryopreserved. This collection time point is called p0.

The AMP cells of the invention are characterized by assaying for physiologically relevant cytokines. Suitable cells are those in which each cytokine occurs in the physiological range of ˜5.0-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2. The cells may optionally be assayed for Thymosin β4.

### Generation of Conditioned Medium (CM)

ECS cell-CM is obtained as described below for ACCS, except that ECS cells are used.

Generation of ACCS—The AMP cells of the invention can be used to generate ACCS. In one embodiment, the AMP cells are isolated as described herein and 1×10⁶ cells/mL are seeded into T75 flasks containing between 5-30 mL culture medium, preferably between 10-25 mL culture medium, and most preferably about 10 mL culture medium. The culture medium is preferably a basal medium (for example IMDM highly enriched basal medium) which is supplemented with human serum albumin as described above. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Collected media is combined to create pools. A preferred pool is a SP pool. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. It is also contemplated that ACCS production be scaled up for generation of sufficient product for clinical testing and for commercialization. It is also contemplated by the invention that ACCS be irradiated. Skilled artisans are familiar with cryopreservation lyophilization, irradiation and sustained-release formulation methodologies.

### Mixed Cell Compositions

In certain embodiments of the invention, the ECS cells, AMP cells, GP-ECS cells, and GP-AMP cells, are mixed with other cell types such as islet cells or islet-like cells. Islet cells can be obtained from a cadaver pancreas as described in the literature (see, for example, Linetsky, E., et al, Diabetes, 1997 Jul;46(7):1120-3). Islet-like cells can be obtained by exposing stem cells, for example ECS cells, including AMP cells, to conditions which cause their differentiation into an islet cell-like phenotype. Details on the differentiation of ECS cells, including AMP cells, into islet-like cells can be found in U.S. Provisional Application No. 61/132,943, incorporated herein by reference in its entirety. In another embodiment of the invention, the ECS cells, including AMP cells, are mixed with GP-ECS cells and/or GP-AMP cells. Details on obtaining GP-ECS cells and GP-AMP cells can be found in U.S. Provisional Application No. 61/132,943, incorporated herein by reference in its entirety. In still other embodiments, the ECS cells, including AMP cells, can be mixed with any cell type capable of secreting insulin to create the combination compositions useful in practicing the methods of the invention.

### Genetic Engineering

In yet another embodiment of the invention, the ECS cells or AMP cells may be genetically engineered to produce human insulin. Methods which are well known to those skilled in the art can be used to construct expression vectors containing a nucleic acid encoding the human insulin gene linked to appropriate transcriptional/translational control signals. The sequence of human insulin is readily obtainable by accessing the public NCBI Nucleotide Database at the following ur1: http://www.ncbi.nlm.nih.gov/sites/entrez and searching the database using the following number: NM_{—}000207. Alternatively, a cDNA clone of the human insulin gene may be ordered using the following information: Clone: MGC:12292 (IMAGE:3950204), Clone Sequence: BC005255.1, Vector: pDNR-LIB, Corresponding RefSeq, mRNA: NM_{—}000207.2 from American Type Culture Collection, RZPD German Resource Center for Genome Research, Geneservice Ltd, Harvard Institute of Proteomics, or Open Biosystems. Once the expression vector is constructed, skilled artisans may use familiar techniques to transfect the cells and isolate resulting human insulin-secreting cells.

### Formulation

Formulations suitable for topical administration in accordance with the present invention comprise therapeutically effective amounts of the therapeutic agent with one or more pharmaceutically acceptable carriers and/or adjuvants. ECS cells, ECS cell-CM, AMP cells, ACCS, and/or cell lysates or cell products derived from the cells, each in combination with insulin, may be used in conjunction with a variety of materials routinely used in the treatment of wounds, such as collagen based creams, films, microcapsules, or powders; hyaluronic acid or other glycosaminoglycan-derived preparations; creams, foams, suture material; and wound dressings. Alternatively, the ECS cells, ECS cell-CM, AMP cells, ACCS, and/or cell lysates or cell products derived from the cells, each in combination with insulin, can be incorporated into a pharmaceutically acceptable solution designed for topical administration.

The compositions of the invention can be prepared in a variety of ways depending on the intended use of the compositions. For example, a composition useful in practicing the invention may be a liquid comprising an agent of the invention in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even both viscous and muco-adhesive.

### Pharmaceutical Compositions

The present invention provides pharmaceutical compositions in a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

### Administration

The combination compositions of the invention are applied in a therapeutically effective amount to accomplish accelerated wound healing, including increased wound strength and decreased wound failure, in particular, in chronic wounds. A “therapeutically effective amount” of a therapeutic agent within the meaning of the present invention will be determined by a patient's attending physician or veterinarian. Such amounts are readily ascertained by one of ordinary skill in the art and will enable accelerated wound healing when administered in accordance with the present invention. Factors which influence what a therapeutically effective amount will be include, the specific activity of the therapeutic agent being used, the wound type (mechanical or thermal, full or partial thickness, acute or chronic, etc.), the size of the wound, the wound's depth (if full thickness), the absence or presence of infection, time elapsed since the injury's or infliction or onset, and the age, physical condition, existence of other disease states (i.e. diabetes), and nutritional status of the patient. Additionally, other medication the patient may be receiving will effect the determination of the therapeutically effective amount of the therapeutic agent to administer.

In a preferred embodiment of the present invention. ECS cells, ECS cell-CM, AMP cells, ACCS, and/or cell lysates or cell products derived from the cells, each in combination with insulin, should be topically administered to the wound site to promote accelerated wound healing in the patient. This topical administration can be as a single dose or as repeated doses given at multiple designated intervals. It will readily be appreciated by those skilled in the art that the preferred dosage regimen will vary with the type and severity of the injury/wound being treated.

### Dosage

One of skill in the art may readily determine the appropriate concentration, or dose, of the combination compositions for a particular purpose. The skilled artisan will recognize that a preferred dose is one which produces a therapeutic effect, such as accelerating chronic wound healing, in a patient in need thereof.

One of skill in the art may readily determine the appropriate concentration, or dose, of the cells, for example, AMP cells or ACCS, for a particular purpose. The skilled artisan will recognize that a preferred dose is one which produces a therapeutic effect, such as treating chronic wounds in a patient in need thereof. For example, ECS cells or AMP cells are prepared at a concentration of between about 1×10⁷-1×10⁸ cells/mL, preferably at about 2.5×10⁷-7.5×10⁷ cells/mL, and most preferably at about 5×10⁷ cells/mL. The volume of cell mixture administered will depend upon several variables and can only be determined by the attending physician at time of use. In addition, one of skill in the art may readily determine the appropriate concentration of ECS cell-CM or ACCS for a particular purpose. A preferred dose is in the range of about 0.5-2000 μL/cm². Another preferred dose is in the range of about 5-1000 μL/cm². Another preferred dose is in the range of about 50-100 μL/cm². In a particularly preferred embodiment, it has been found that relatively small amounts of ACCS can accelerate wound healing. Of course, proper doses of ECS cells, AMP cells, ECS cell-CM, or ACCS will require empirical determination at time of use based on several variables including but not limited to the severity and type of disease, injury, disorder or condition being treated; patient age, weight, sex, health; other medications and treatments being administered to the patient; and the like. One of skill in the art will also recognize that number of doses (dosing regimen) to be administered needs also to be empirically determined based on, for example, severity and type of disease, injury, disorder or condition being treated. In a preferred embodiment, one dose is sufficient. Other preferred embodiments contemplate, 2, 3, 4, or more doses.

The amount of insulin used in the combination compositions should be within normal physiologic ranges for fasting insulin levels. For example, a suitable dose may be in the range of about 2.0-20.0 μIU/mL.

### Treatment Kits

The invention also provides for an article of manufacture comprising packaging material and a pharmaceutical combination composition of the invention contained within the packaging material, wherein the pharmaceutical combination composition comprises combination compositions of ECS cells, AMP cells, ECS cell-CM, ACCS, or cell lysates and cell products derived from the cells, each in combination with insulin (i.e. ECS cells/insulin; ECS cell-CM/insulin; ECS cell lysates/insulin; ECS cell products/insulin; AMP cells/insulin; ACCS/insulin; AMP cell lysates/insulin; AMP cell products/insulin) or in combination with each other as well as in combination with insulin (i.e. ECS cells/ECS cell-CM/insulin, etc.). In addition, the kits may also comprise a pharmaceutical composition comprising GP-ECS cells, GP-AMP cells, GP-ECS cell-CM, or GP-ACCS. The packaging material comprises a label and/or package insert which indicates that the combination compositions can be used for treating wounds.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Recovery of AMP cells—AMP cells were dissociated from starting amniotic membrane using the dissociation agents PXXIII. The average weight range of an amnion was 18-27 g. The number of cells recovered per g of amnion was about 10-15×10⁶.

Method of obtaining selected AMP cells—Cells were plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in IMDM basal medium supplemented with 0.5% human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Generation of ACCS

The AMP cells of the invention can be used to generate ACCS, including pooled ACCS. The AMP cells were isolated as described above and ˜1×10⁶ cells/mL were seeded into T75 flasks containing ˜10 mL culture medium as described above. The cells were cultured until confluent, the medium was changed and ACCS was collected 3 days post-confluence. Optionally, the ACCS is collected again after 3 days, and optionally again after 3 days. Collected media are combined to make pools. Skilled artisans will recognize that other embodiments for collecting ACCS from confluent cultures, such as using other tissue culture vessels, including but not limited to cell factories, flasks, hollow fibers, or suspension culture apparatus, etc. are also contemplated by the methods of the invention (see Detailed Description above). It is also contemplated by the instant invention that the ACCS be cryopreserved, lyophilized, irradiated or formulated for sustained-release following collection. It is also contemplated that ACCS be collected at different time points (see Detailed Description for details).

### Example 3

### Generation of Pooled ACCS

ACCS was obtained essentially as described above. In certain embodiments, ACCS was collected multiple times from an AMP cell culture derived from one placenta and these multiple ACCS collections were pooled together. Such pools are referred to as “SP pools” (more than one ACCS collection/one placenta). In another embodiment, AMP cell cultures were derived from several placentas, i.e. from 5 or 10 placentas. The AMP cells from each placenta were cultured and one ACCS collection from each culture was collected and then they were all pooled. These pools are termed “MP1 pools” (one ACCS collection/placenta, multiple placentas). In yet another embodiment, AMP cell cultures were derived from several placentas, i.e. from 5 or 10 placentas. The AMP cells from each placenta were cultured and more than one ACCS collection was performed from each AMP cell culture and then pooled. These pools are termed “MP2 pools” (more than one ACCS collection/placenta, multiple placentas).

### Example 4

### Preparation of Combination Compositions

The following combination compositions are prepared in a suitable carrier:

ECS cells+insulin

ECS cells genetically modified to secret insulin

ECS cells+insulin-secreting islet cells

ECS cells+insulin-secreting islet-like cells

AMP cells+insulin

AMP cells genetically modified to secret insulin

AMP cells+insulin-secreting islet cells

AMP cells+insulin-secreting islet-like cells

ECS cell-CM+insulin

ECS cell-CM+insulin-secreting islet cells

ECS cell-CM+insulin-secreting islet-like cells

ACCS+insulin

ACCS+insulin-secreting islet cells

ACCS+insulin-secreting islet-like cells

ECS cells+GP-ECS cells

ECS cells+GP-AMP cells

AMP cells+GP-AMP cells

AMP cells+GP-ECS-cells

ECS cell-CM+GP-ECS cells

ECS cell-CM+GP-AMP cells

ACCS+GP-ECS cells

ACCS+GP-AMP cells

Although not specifically set forth, it should be noted that other similar and equivalent combination compositions are contemplated by the methods of the invention.

ECS cells, ECS cell-CM, AMP cells, ACCS, islet cells, islet-like cells, GP-ECS cells, GP-AMP cells, GP-ESC-CM and GP-ACCS are obtained as described elsewhere in the specification. Insulin may be obtained from Novo-Nordisk, product name Novolin-N Insulin U-100, 10 mL Vial, catalog #169183411. Suitable carriers are described elsewhere in the specification.

### Example 5

### Effects of Combination Compositions in an Animal Model of Chronic Wound Healing

An art-accepted animal model for chronic granulating wound is used to study the effects of the combination compositions described in Example 4 on chronic wound healing (Hayward PG, Robson MC: Animal models of wound contraction. In Barbul A, et al: Clinical and Experimental Approaches to Dermal and Epidermal Repair: Normal and Chronic Wounds. John Wiley & Sons, New York, 1990.). Doses used are described elsewhere in the specification.

### Example 6

### Effects of Combination Compositions in an Animal Model of Diabetic Ulcers

An art-accepted animal model for wound healing in db/db mice is used to study the effects of the combination compositions described in Example 4 on chronic wound healing (Sullivan, S. R., et al., Validation of a model for the study of multiple wounds in the diabetic mouse (db/db) Plast Reconstr Surg 2004, Mar;113(3):953-60). Doses used are described elsewhere in the specification.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A method of accelerating the rate of healing of a chronic wound in a subject in need thereof comprising applying to the chronic wound a combination composition selected from the group consisting of a combination composition comprising Extraembryonic Cytokine-Secreting (ECS) cells, insulin, and a carrier; a combination composition comprising conditioned media derived from ECS, insulin, and a carrier; a combination composition comprising Amnion-derived Multipotent Progenitor (AMP) cells, insulin, and a carrier; a combination composition, and a combination comprising Amnion-derived Cellular Cytokine Solution (ACCS), insulin, and a carrier, wherein the chronic wound is selected from the group consisting of a diabetic ulcer, a venous ulcer, a pressure ulcer, and a sickle cell ulcer.

2. A method of lowering glucose concentrations in chronic wound fluid in a subject in need thereof comprising applying to the chronic wound a combination composition selected from the group consisting of a combination composition comprising Extraembryonic Cytokine-Secreting (ECS) cells, insulin, and a carrier; a combination composition comprising conditioned media derived from ECS, insulin, and a carrier; a combination composition comprising Amnion-derived Multipotent Progenitor (AMP) cells, insulin, and a carrier; a combination composition, and a combination comprising Amnion-derived Cellular Cytokine Solution (ACCS), insulin, and a carrier, wherein the chronic wound is selected from the group consisting of a diabetic ulcer, a venous ulcer, a pressure ulcer, and a sickle cell ulcer.

3. The method of claim 1 or 2 wherein the insulin in the composition is added to the composition.

4. The method of claim 1 or 2 wherein the insulin in the composition is added to the composition by ECS cells or AMP cells which have been genetically modified to secrete insulin.

5. The method of claim 1 or 2 wherein the insulin in the composition is added to the composition by combining ECS cells or AMP cells with insulin-secreting islet cells or insulin-secreting islet-like cells.

6. The method of claim 1 or 2 wherein the combination composition is selected from the group consisting of:
Composition A: ECS cells+GP-AMP cells;
Composition B: ECS cells+GP-AE cells;
Composition C: AMP cells+GP-AMP cells;
Composition D: AMP cells+GP-AE cells;
Composition E: ECS conditioned media+GP-AMP cells;
Composition F: ECS conditioned media+GP-AE cells;
Composition G: ACCS+GP-AMP cells; and
Composition H: ACCS+GP-AE cells.
